(19) United States
(12) Patent Application Publication
(10) Pub. No.: US 2007/0141567 A1
(43) Pub. Date: Jun. 21, 2007

(54) Waveguide system for detection of fluorescently labeled nucleic acid sequences

(76) Inventors: David R. Wulfman, Minneapolis, MN (US); Tracey L. Baas, Seatlle, WA (US)

Correspondence Address: GRAY, PLANT, MOOTY, MOOTY & BENNETT, P.A., P.O. BOX 2906, MINNEAPOLIS, MN, 55402-0906, US

(21) Appl. No.: 10/534,625

(22) PCT Filed: Nov. 13, 2003
(86) PCT No.: PCT/US03/36146

§ 371 Date: Nov. 6, 2006

Related U.S. Application Data
(60) Provisional application No. 60/426,329, filed on Nov. 13, 2002.

(51) Int. Cl. C12Q 1/68; C12M 3/00; G01N 33/543

(52) U.S. Cl. 435/6; 435/287.1

(57) ABSTRACT

A system for detecting and identifying fluorescently labeled nucleic acid sequences. An embodiment of the system is comprised of an optical waveguide, an excitation light, a photo detector, filters for select fluorescent emissions, a reaction chamber, a data interpretation algorithm, and an addressable array, which assigns a known nucleic acid sequence at a precise location on the waveguide. An embodiment of the system uses a glass slide based array as an optical waveguide. Nucleic acid sequences are immobilized or hybridized to the surface of the waveguide. This abstract is intended to comply with the rules requiring an abstract that will allow a searcher or other reader to quickly ascertain the subject matter of the technical disclosure. It is submitted with the understanding that it will not be used to interpret or limit the scope of meaning of the claims.

### RELATED APPLICATIONS

The priority date of provisional patent application Ser. No. 60/426,329, filed Nov. 13, 2002 is claimed and the application is incorporated by reference into this specification.

### FIELD OF TECHNOLOGY

The technology described in this specification is in the field of detection of fluorescently labeled nucleic acid sequences. More particularly, the system detects and identifies nucleic acid sequences immobilized or hybridized to the surface of a waveguide.

### Introduction

This specification describes a system for detecting and identifying fluorescently labeled (sometimes referred to as “tagged” or “marked”) nucleic acid sequences. An embodiment of the system uses a glass slide (for example, a microscope slide) based array as an optical waveguide. Nucleic acid sequences are immobilized or hybridized to the surface of the waveguide. The system is comprised of the optical waveguide 2, an excitation light 1, a photo detector 3, filters for select fluorescent emission 4 and 5, a reaction chamber, and a data interpretation algorithm. The optical waveguide detector is a low density fluorescent array detector. An embodiment of the waveguide performs detection of multi-analytes. No image gathering or analysis is required. The waveguide detector does not require a specially patterned waveguide surface for detection. It also comprises an automated readout algorithm. The optical waveguide also is comprised of an addressable array, which assigns a known nucleic acid sequence at a precise location on the waveguide.

The system is useful in the field of molecular diagnostics. It is used in the clinical medicine laboratory for nucleic acid testing for patient care. Genetic testing involves the examination of nucleic acid from patient samples taken to render a medical diagnosis. The diagnosis is based on the identification of one or more specific DNA sequences or RNA transcripts characterizing specific diseases. Most of the diseases understood at the gene level involve alterations in specific nucleotides. When present, the sequence alterations or mutations result in change in specific proteins, which in turn alter the function of a cell.

Prior to identification of nucleic acid, a sample of the patient's genetic material is collected. Although, testing of human patients is the most prevalent application of genetic testing, genetic testing of other organic material is also prevalent. The subject of the test may be animal, plant, or any other thing living or once living. Blood, for example, is a common sample taken from an animal source. The collected sample is purified. Purification is the process of removal of the unwanted constituent parts of the sample. It is relatively easy to remove unwanted parts of whole blood by fractionating it into its constituent parts, one of which is genetic material (DNA and RNA). The genetic material is amplified to increase the amount of genetic material to a quantity sufficient to carry out testing. In fluorescence detection of nucleic acids, a fluorescent molecule (marker, tag) must be associated with the sequences being sought in a sample (the targets). This tag association can be accomplished at different stages in sample processing, either during amplification/discrimination (like Polymerase Chain Reaction, PCR, or other process) or during a subsequent process (Lipase Detection Reaction, LDR e.g.). Typically, the fluorescently tagged targets, which are single stranded DNA (oligonucleotides, oligos), are then hybridized to complimentary oligos (probes) that are immobilized in a specific region on a surface. Hybridization occurs when one of the ends of the target become attached to one of the free ends of the oligonucleotide probes. The oligonucleotide probes are made up of a single stranded genetic sequence complementary to a target. If the fluorescently labeled target that is complimentary to a given probe is present, under the correct processing conditions it hybridizes to that probe. The region of the surface where that probe has been immobilized then can be fluorescently excited due to this hybridization event between probe and fluorescently labeled complimentary target. This process is regulated by the addition of chemical reagents in a thermally controlled aqueous environment. Since each specialized probe is immobilized within a hybridization chamber, on a hybridization substrate, or within or on some other hybridization structure, the coordinates of each individual probe are definable. Consequently, multiple DNA sequences can be simultaneously queried within the chamber, on the substrate, or in some other hybridization structure on which the probe is immobilized.

Current methods for analyzing gene sequences involves the detection of fluorescence from DNA immobilized on a solid surface when the immobilized DNA is exposed to a solution containing a nucleic acid sample (test solution) whose makeup is at least partially unknown. When many sequences are analyzed simultaneously a DNA micro array is employed. Micro arrays are typically read by a laser array scanner. Scanners consist of lasers that excite the array and a photo sensor, typically a photo multiplier tube, that records any resulting fluorescent emission. The scanner then generates an image of the fluorescent spots on the array as generated from emission wavelength alone. In this way, one can determine the fluorescent landscape of the array. A similar method uses both fluorescent microscopy and evanescent microscopy techniques. However in place of a scanning laser incrementally exciting portions of the array, microscopes ablate the entire array, the image of which is recorded by a camera, or charge coupled device.

Other detection systems also rely on binding probes to a specific site on the surface of an array. An example of a system includes an array designed to function as a waveguide as well as a support for the probe. The system detects light scattered at a surface of the waveguide. The light source illuminates the entire waveguide through an edge of the waveguide, rather than relying on fluorescent excitation.

There are also fluorescent detection systems that use waveguides in combination with a stationary excitation source to deliver energy in the form of an evanescent wave. The entire surface of the waveguide is simultaneously flooded with the evanescent wave. In other words, the excitation energy is not selectively delivered to specific sites, i.e., discrete areas of the waveguide. The detection methods of these systems are typically limited to copying an instantaneous image of the entire array surface or to rapid sequential scanning of the array surface to gather images of discrete areas of the array surface for later collation into a composite image of the surface.

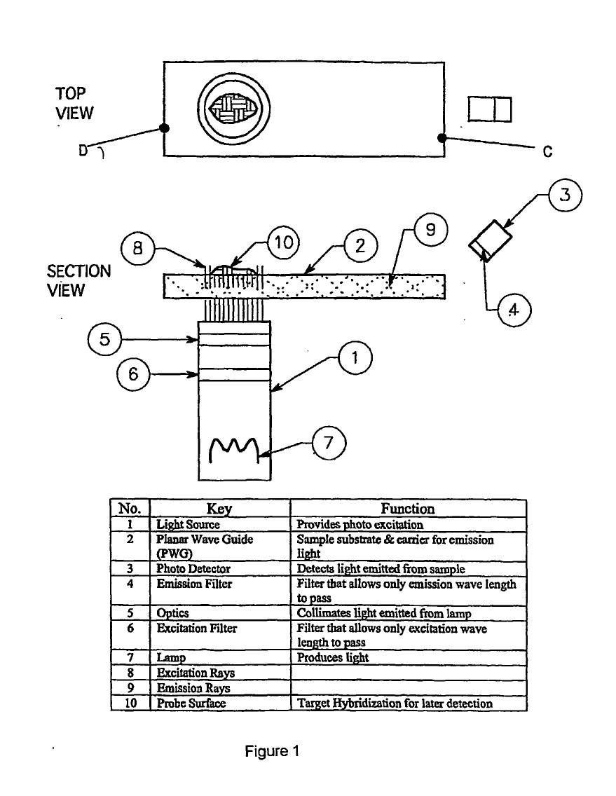

A simpler and lower cost alternative to these methodologies is the system described in this specification. The system relies on excitation of specific regions of a waveguide surface and detection of any resulting emission generated from fluorescently labeled substrate that might be located there. In FIG. 1, excitation light source 1 is positioned such that its illumination path is perpendicular to the DNA bound to the surface of waveguide 2, i.e., a surface of the microscope slide. If fluorescent material is located in the illuminated region, a portion of its emitted fluorescent light enters waveguide 2 at a sufficiently shallow angle to permit propagation within it. This propagation occurs because of the phenomenon known as total internal reflection. A portion of the propagated light exits waveguide 2 at one of its ends, where photo detector 3 is positioned to sense it.

This waveguide 2 embodiment exploits total internal reflection fluorescence transmission such that multiple analytes in a fluorescent array of potential binding sites can be collocated on the same waveguide 2 surface (the “substrate”) and be separately distinguished. This is achieved by continuously monitoring fluorescence output from the waveguide's end while simultaneously exposing its surface to a moving excitation light source 1. Collected data show waveguide output as a function of excitation light source 1 position relative to the waveguide's edge, some other feature of waveguide 2, or some feature of the array itself.

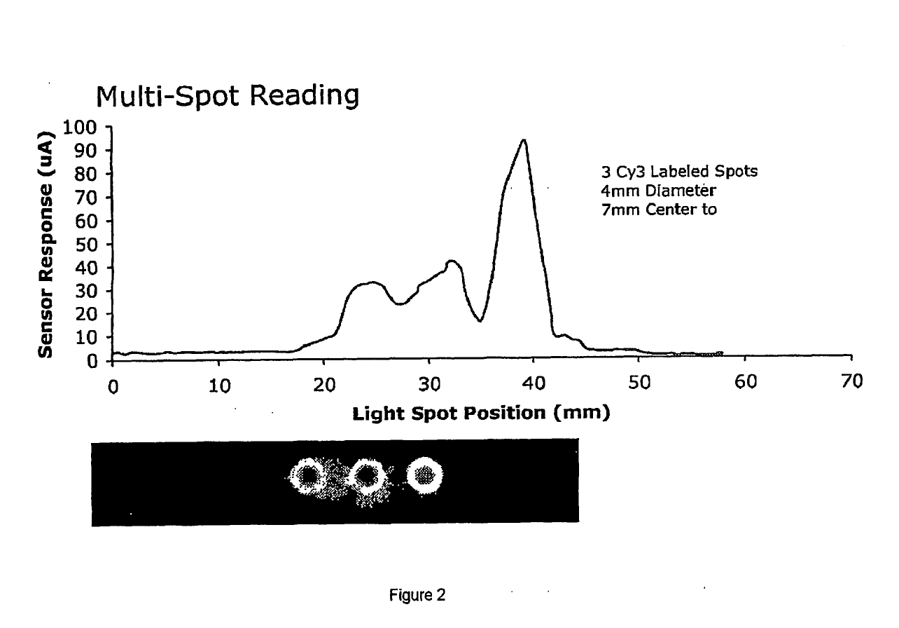

A typical output of the planar waveguide system is shown in the plot of FIG. 2. Under the plot is a laser scanned image of the same array. It is important to note that not only are the spots distinguished from one another, but the intensities of their output can be quantified and compared using the planar waveguide. This kind of quantification enables application of bioinformatics and automated analysis in diagnosis, which can reduce the overall cost of a given test. Clinical samples collected for a common genetic disorder, the Factor V Leiden mutation, were compared using the optical waveguide system to readings taken from a laser scanner. Of the 20 samples examined in this preliminary test, there was over a 98% correlation of the results from the waveguide and from those of a laser scanner.

Although the fluorescence detection method discussed here can be resolved by visual inspection of data plotted similarly to that shown in FIG. 2, an automated interpretation of the data is more practical. Such automation relieves the user from making determinations by eye, reducing the risk of array misidentification, inconsistency between users, and intensity measurement error.

An embodiment of the detection and identification system described in this specification also comprises a data interpretation algorithm. One embodiment of the algorithm is tailored for two-dimensional arrays. Another embodiment is tailored for one-dimensional arrays, which is a special case of the two-dimensional arrays. One of ordinary skill in the art could modify the algorithm for a 3-dimensional array. The data interpretation algorithms quickly and accurately resolve signal overlap between spots and provide intensity data that can be compared between loci.

An embodiment of the system detects and/or identifies multiple analytes by reading fluorescent arrays, also known as fluorescent micro arrays, arranged in an array format with a surface on which a probe for the analyte (e.g., DNA and oligonucleotides) is bound to one or more specific sites. Detection of the analyte is performed by contacting the probe with the analyte to be detected. The specific site of the array on which the probe is located is then excited with a fluorescent energy source, which causes the probe to fluoresce if the analyte that is the complement of the probe is present. Confirmation of the presence of the analyte and its identification is obtained by analyzing the fluorescent emission associated with the site on the array to which the probe is bound. The analyte's concentration may also be determined from the measure of that emission.

Most extant detection methods rely on pattern recognition of images taken of micro arrays or liquid well arrays that contain DNA or oligonucleotides. Unlike those methods, the embodiments of the waveguide detection system described in this specification expose probe surfaces to a specific wavelength of light (excitation light), which is absorbed by the fluorescent tags (fluors) in the probes. The fluors then re-emit the light at a longer wavelength (emission light), which is detected by a properly tuned photo detector. If the photo detector detects emission from a given probe surface, the DNA sequence associated with it is present in the organic organism from which the sample was taken.

### DESCRIPTION OF EMBODIMENTS

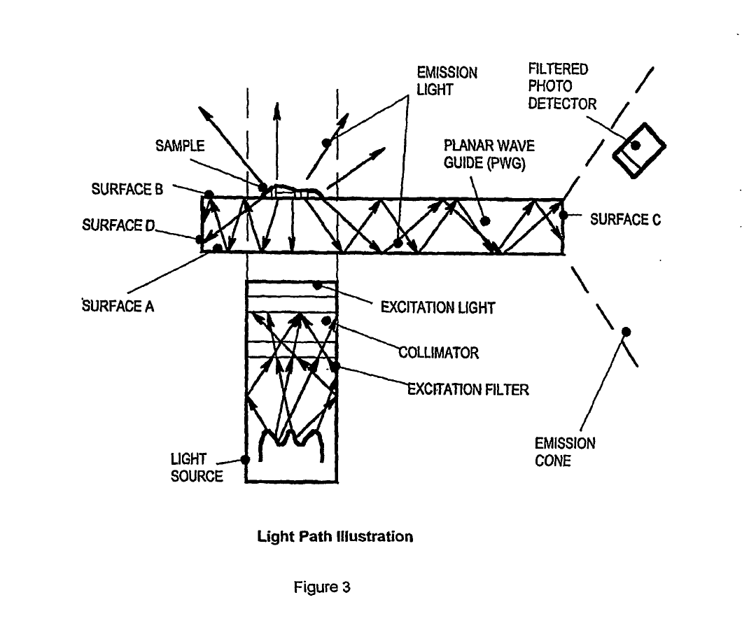

An embodiment of a waveguide detection system is illustrated in FIGS. 1 and 3. FIGS. 1 and 3 illustrate a planar waveguide detection system. However, other geometries of the waveguide form alternative embodiments of the waveguide detection system described in this specification. These other geometric waveguide embodiments do not depart from the technological concepts described in this specification. For example, a cylindrical waveguide may be substituted for the planar waveguide and function to detect and identify genetic material in a sample of organic matter. Adaptation of the waveguide detection system to different waveguide geometry may be accomplished by a person of ordinary skill in the art.

FIG. 1 shows the basic layout of an embodiment of the planar waveguide detection system. It is comprised of planar waveguide 2, excitation light source 1, and photo detector 3. Probes 10 are immobilized on planar waveguide 2. Probes 10 can hybridize to a specific DNA target sequence. The immobilized probes 10 coincide directly with light source 1 output. In one embodiment, light source 1 is comprised of lamp 7, excitation filter 6, and optics 5. In the embodiment, photo detector 3 has an emission filter 4 and is aligned with planar waveguide 2 to receive the maximum amount of light emitted from waveguide 2. Photo detector 3 may be a photodiode, a photo multiplier tube, or other photo sensitive transducer.

FIG. 3 illustrates the various paths of the fluorescent light generated by probes 10 when they are hybridized with their fluorescently label complement in the presence of photo excitation light 1. Planar waveguide 2 is comprised of three active surfaces. Surfaces A and B are opposed parallel surfaces. FIG. 3 illustrates surface A as the planar surface into which excitation light 1 enters the waveguide. The waveguide system may, however, be configured so that excitation light 1 enters surface B or another surface of waveguide 2. Surface B is the planar surface on which probes are bound. Light traveling through waveguide 2 exits out of an edge of the waveguide at surface C where it is detected by photo detector 3. Surface B binds at least one and preferably a plurality of analyte probes to specific sites on the surface.

Excitation light source 1 is mounted with respect to planar waveguide 2 in a direction generally normal to surface A of waveguide 2. As previously noted, light source 1 may also be mounted in a direction normal to surface B. The orientation normal to surface A propagates the excitation energy through surface A, through thickness 11 of waveguide 2, through surface B, and through the surface of probes 10 bound to a specific binding site or sites located on surface B. Light source 1 produces a dispersed pattern of white light. The dispersed white light is transmitted through excitation band pass filter 6. Band pass filter 6 allows a relatively narrow frequency band of light to pass through it. Within the passed band is the wave length that excites the selected fluorescent probe. The light band allowed to pass is further transmitted through collimating optic 5, which emits only light that is perpendicular to surfaces A and B of planar waveguide 2. Collimation ensures that the majority of the light entering planar waveguide 2 passes through waveguide 2 and strikes probe 10 instead of propagating by internal refraction to other locations in waveguide 2. Collimation maximizes excitation of probe 10 for a given light source and minimizes propagation of light noise through waveguide 2.

For one or more probes 10 on binding site 12 which hybridizes with a fluor labeled analyte which is the complement of probe 10, the fluorescent label associated with the complement absorbs the excitation light and re-emits fluorescent light at a longer wave length (the emission wave length) in response to the occurrence of the (i) complement that hybridizes to the probe and (ii) excitation by light of a wavelength corresponding to the absorbsion wavelength of the dye. The fluorescent light energy is emitted, as shown in FIG. 3, in a scattered pattern. Some of the fluorescent light travels into the medium surrounding waveguide 2 and is lost. The remainder enters through surface B of waveguide 2. A portion of the fluorescent light entering waveguide 2 will have an angle of incidence with surface A that causes it to pass through surface A and also escape into the medium surrounding waveguide 2. The remaining portion is trapped inside waveguide 2 by internal refraction and propagates within waveguide 2 by total internal reflection in a net direction parallel to surfaces A and B until it reaches an edge, i.e., surface C in FIG. 3, of waveguide 2. A portion of the propagated light exits surface C where it is detected by photo detector 3, which is located proximate edge surface C. Photo detector 3 transduces the light into an electric current. Production of an electric current signal by detector 3 indicates that (i) the target hybridized to probe surface B at its specific binding site 12 and (ii) the DNA sequence that is complementary to probe 10 is present in the sample. Conversely if no fluor labels are present on probe surface 10, then no emission light will propagate through waveguide 2 and photo detector 3 will not be excited.

Although FIG. 1 shows excitation filter 6 on photo excitation light source 1 and emission filter 4 on photo detector 3, these may be eliminated. They can be eliminated by photo isolation between probe surface 10 and photo detector 3. Collimation of the light source 1 by optic 5 makes their elimination possible. Since the collimated excitation light enters waveguide 2 perpendicular to surfaces A and B, the, majority of the excitation light passes directly through waveguide 2 and out surface B. The rays coincident with the probe surface are absorbed by it and are either re-emitted in a dispersed pattern as heat or as the emission wavelength associated with the flurochrome label of the target hybridized to it. This leaves very little, if any, of the excitation light to actually propagate through waveguide 2 and excite photo detector 3. In other words, whatever light actually excites photo detector 3, if any, comes from emission of the fluorochromes and not from light source 1. In effect, the combination of collimating, orientation, and the geometry of the waveguide serves as a filtration system itself.

The embodiments in this specification indicate the presence or absence of a hybridized target without the need for imaging. Imaging is a common means employed for confirming the presence or absence of DNA sequences in a sample, but it requires either a skilled person or an automated system to read and interpret the images. The embodiments in this specification do not require a skilled person or an automated system.

Excitation source 1, located normal to and directed at surface A, moves over fixed waveguide 2 to scan each specific probe site. Alternatively, excitation source 1 is fixed and waveguide 2 moves over excitation source 1 to scan each specific probe site. In another alternative, excitation source 1 is optically scanned across surface B of waveguide 2, e.g., by using mirrors, optical fibers, and waveguides. A scanning motion embodiment is comprised of iterations of movement in a straight line down a column, to another column, and then down that column. This embodiment can be implemented by the same process, but from row to row. Another scanning movement embodiment is a zig-zag pattern of almost any sort. Another scanning embodiment is based upon some random scanning algorithm. Another alternative embodiment of the planar waveguide process is a cylindrical waveguide. The foregoing scanning embodiments may also be used with the cylindrical waveguide embodiment. Another embodiment for the cylindrical waveguide-embodiment scanning method is scanning along a circumferential segment, movement to another circumferential segment, and then scanning that segment and so forth. A further scanning embodiment is movement along a helical segment on a cylindrical waveguide and then to other parallel helical segments on the cylinder.

The fluorescent energy signal from the waveguide detector is processed by a specialized signal processing device or by a general purpose computing device. The fluorescent energy signal is processed based upon a variety of parameters such as wavelength and signal strength. The photo detector 3 signals are correlated to the position of the origin of the excitation energy on waveguide 2. This is necessary to establish the nature of the probe on the binding site emitting the detected fluorescent energy. The nature of the probe in turn provides the nature of the genetic material that hybridized to the probe.

An embodiment of the waveguide detector includes specific binding sites 11 spaced apart from each other on the array in the same direction as excitation source 1 is scanned across waveguide 2. In other embodiments there are multiple photo detectors 1 positioned along edge surface C of waveguide 2 to simultaneously detect fluorescent energy signals from a plurality of binding sites 1.

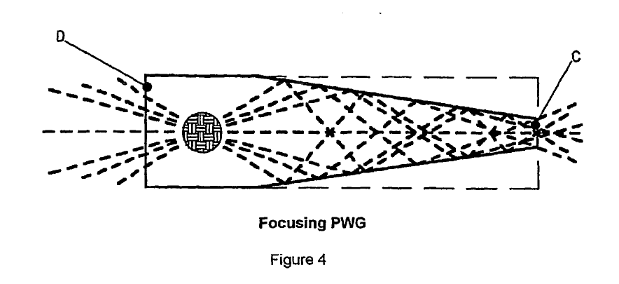

Although the top view of FIG. 1 shows planar waveguide 2 as a rectangle, the actual shape of the surfaces A and B can be configured such that the light exiting surface C is more focused and intense. As illustrated in FIG. 3, the long walls of the planar waveguide 2 can converge making surface C narrower than surface D. If the angle of convergence is small enough to effect total internal reflection, then the light propagating toward surface C is condensed. The same flux of light exits through surface C in FIGS. 1 and 4, but because the area of surface C shown in FIG. 4 is smaller, its light is more intense. With the appropriate placement of a small photo detector (like a photodiode), a greater percentage of the actual light emitted from the fluors is channeled for detection. The light rays shown in FIG. 4 are depicted for the purpose of concept illustration, and do not depict the total pattern of light dispersed from the probe surface shown.

Detection of multiple DNA sequences from the same sample using multiple probe surfaces is a feature that makes identification of genetic material fast and more economical. For purposes of this specification this feature is referred to as multiplexing. Multiplexing may be accomplished in a number of ways. One way is to package separate planar waveguides together so their probe surfaces share the same reaction chamber or substrate, yet be sufficiently photo isolated from one another to avoid crosstalk during detection. Another way of implementing multiplexing is to place multiple probe surfaces on the same planar waveguide. The waveguide is configured to photo isolate the multiple probe surfaces from one another to avoid crosstalk.

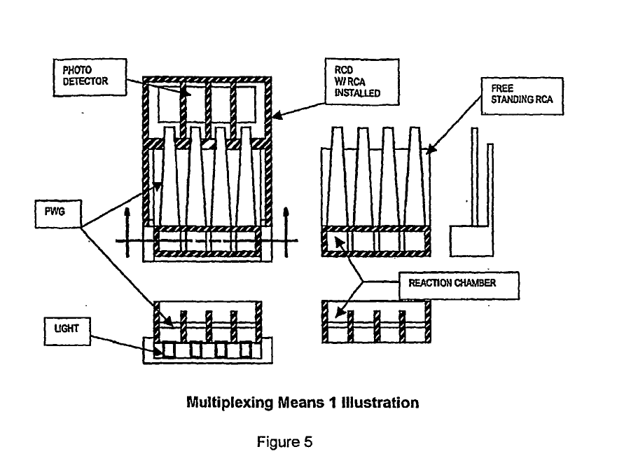

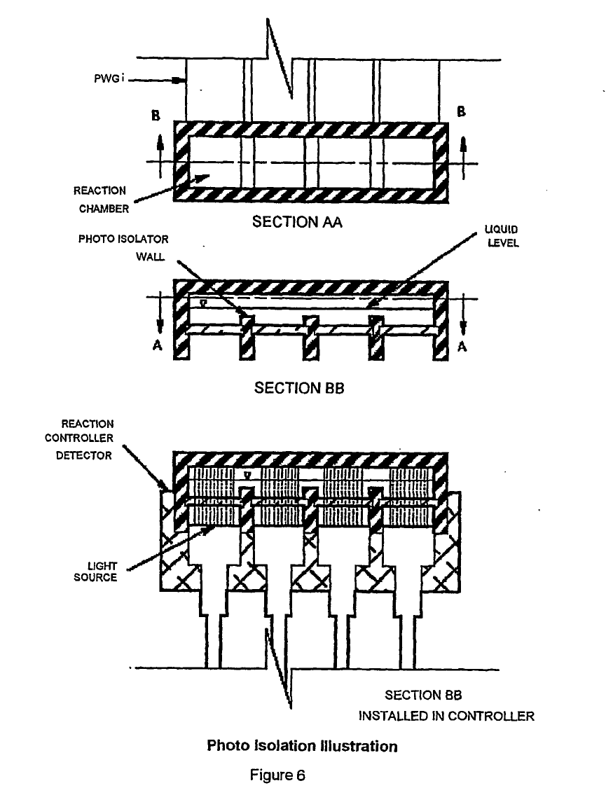

Both of the multiplexing systems shown in FIGS. 5 and 6 share structural features in common. There are one or more planar waveguides mounted in a structure. The structure provides support, protection, and alignment of waveguides 2. The combination of waveguide 2 and its support structure is referred to as a reaction chamber 20. The reaction chamber is disposable. Like sharps, it is designed to be used only once for analysis of a single biological sample. The process of identification of genetic sequences from a sample includes, as previously stated, purification, amplification, and hybridization of the genetic sequences.

A second apparatus, the reaction controller/detector, is a reusable device for controlling reaction chamber 20, light source 1, photo detector 3, and power source for the reaction or detection processes. The reaction controller/detector is primarily an electronic device. The reaction controller/detector is configured to mate with reaction chamber 20 in such a manner that the probes are aligned with light source 1 and the photo detector 3 is aligned with the output of planar waveguide 2.

FIG. 5 illustrates an embodiment of planar waveguide multiplexing. The floor of reaction chamber 20 is a series of glass plates held together in an opaque plastic matrix. The plates are configured so that a first end is a portion of reaction chamber 20 floor and a second end extends outside chamber 20. A photo detector 3 is positioned relative to the second end for receiving emission light from the second end. An individual glass plate bears a specific probe associated with a given DNA.sequence. If that sequence is present in the sample and the associated complementary fluorescent target hybridizes to it, then the fluor will produce emission light when exposed to the proper excitation light. Since the plate is optically isolated from the other plates making up the reaction chamber 20 floor, it is the only plate through which emission light emanating from its probe surface travels. Due to its optical isolation, no other emission light from the surface of a probe on another plate in the chamber is able to travel through it. This isolation configuration permits the separate detection of different DNA sequences from the same sample within the same chamber 20.

Excitation may be accomplished either by illuminating the entire floor of chamber 20 with a broad beam excitation source or illuminating individual plates with a more focused source. Detection is achieved through the use of a photo detector like a photodiode or a photo multiplier tube. A single photo detector may be employed to sample the entire device by sequentially aligning it to each individual output surface (for example, surface C shown in FIGS. 1 and 3) and sampling for light. Multiple photo detectors, each aligned to a different plate output surface may also be used to reduce the mechanical complexity of the system by minimizing the number of moving parts and to afford simultaneous sampling of multiple probe/targets.

Photo-isolation between planar waveguides 2 is illustrated in greater detail in FIG. 6. Whether illuminated separately as shown in FIG. 6 or by a larger common light source, each of the probe surfaces is optically isolated from the others by means of the partial height walls shown in section B-B of FIG. 6. Since the walls do not extend above the liquid level of the sample mixture, the probe surfaces share a common fluid source.

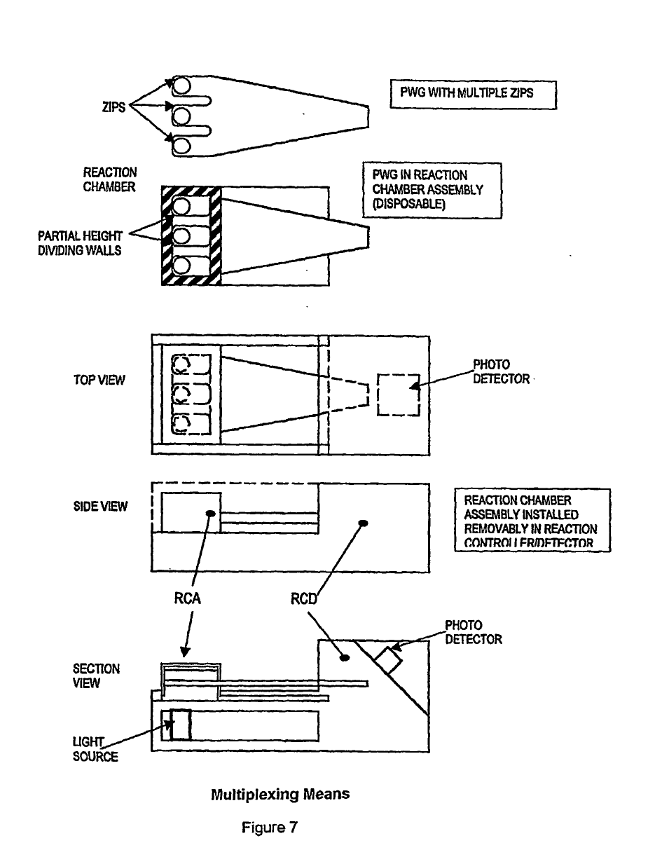

FIG. 7 demonstrates a second multiplexing embodiment. A single glass plate is employed at one end of which is a series of protruding teeth, similar to those of a saw. When integrated in the reaction chamber 20, the teeth are separated from one another photonically, but connected together in a fluidically continuous space. The teeth are separated photonically by walls that function as light blinds. The walls constructed of opaque material, lie between the teeth, and project above the surface of the teeth into chamber 20. The teeth are connected together in a fluidically continuous spate by limiting the height of the walls so they do not project to the top of reaction chamber 20, thereby allowing liquid to flow over the walls and immerse the other teeth, thus keeping each of the teeth fluidically connected. Moreover, the teeth of the plate make up the floor of reaction chamber 20. The opposite end of the glass plate is aligned to photo detector 3. To detect for fluorescence, each tooth is individually and sequentially illuminated by a collimated excitation light source 1. The individual and sequential illumination (and resulting excitation) of one tooth at a time provides clear differentiation and identification of the output of each tooth. If it is known which tooth is illuminated at a given moment, the presence or absence of signal in photo detector 3 at that given time correlates to the presence or absence of the DNA sequence in the sample associated with the probe immobilized on that tooth.

Unlike previously described embodiments which require multiple discrete surfaces for multiplexing, another embodiment requires only a single surface of an optical transmission waveguide on which a known pattern of oligonucleotide spots (probes) are placed. Reducing the detection system to a single waveguide surface reduces the complexity, scale, and part count of a given sensing system. The known pattern of oligonucleotide spots are spaced apart from one another a distance that allows their dual fluorescent signals to be distinguished from one another. As previously described, each spot is composed of unique oligonucleotide probes whose fluorochrome labeled complements are associated with different genetic sequences in a sample. If a target sequence is present in a sample, the fluorochrome labeled complement is liberated into solution. Through mixing and diffusion kinetics, the complement encounters the immobilized oligonucleotide and hybridizes to it. Since a given probe molecule is present in one or a given set of spots on the substrate, only the probe spots whose fluorochrome labeled complements are present in solution will become fluorescently excitable. In this embodiment, immobilized probes are bound to a surface of an optical waveguide. The waveguide is glass or some other medium that permits transmission of electromagnetic energy at wavelengths consistent with those associated with the excitation and emission of the fluorochromes in use.

A cylindrical waveguide embodiment of the waveguide detection system described in this specification was previously mentioned as a means for detecting fluorescently labeled nucleic acids. Use of a cylindrical waveguide for detection also embodies most of the detection techniques described in this specification. The main difference is that instead of detecting for fluorescent regions on the surface of a planar waveguide, this embodiment detects for fluorescent regions on a circular waveguide.

The cylindrical waveguide is also comprised of material that permits transmission of the detected wavelengths of light. Its geometry is circular in cross section (i.e., the section that is perpendicular to the desired direction of light transmission) with either a hollow or solid core. The outer surface of the waveguide is appropriately treated such that it can optimally bear fluorescently labeled nucleic acids either over its entirety or at specific regions. Examples of such surface treatments are silanization and gel coating.

To detect for fluorescence, different regions of the cylindrical waveguide are selectively and specifically exposed to a focused moving light source, the wavelength of which is in the appropriate excitation band. If a specific binding site on the waveguide is fluorescing and the site is in the path of the excitation light, the probe on the binding site produce a fluorescent emission. A portion of the emission will enter the cylindrical waveguide and propagate through it. Fluorescent emission exiting from the cylindrical waveguide's edge end is detected by an appropriately filtered photo detector. The fluorescent landscape of the waveguide is then determined by correlating the level of photo detector excitation with the position of the excitation light source.

The cylindrical waveguide employs the waveguide processes and structure described in this specification and applies them to the cylindrical optical waveguide. The circular cross section of the cylindrical waveguide may be solid or hollow. Fluorescent loci are placed on the outside surface of an embodiment of the waveguide in the form of rings around its circumference, by direct application or through a biochemical assay. As with the planar waveguide, if a given ring is illuminated with the proper wavelength-of excitation light, it will produce a fluorescent emission, which in part is absorbed within the waveguide.

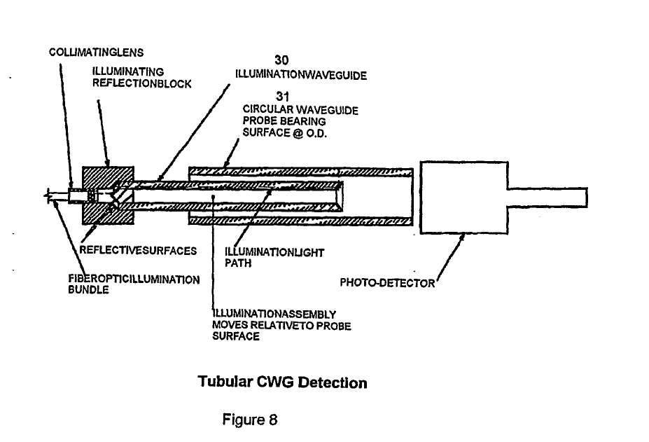

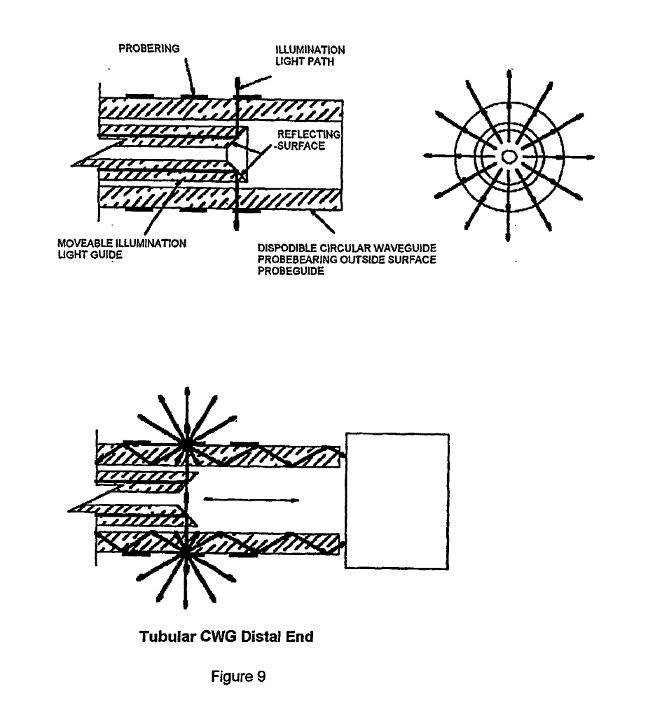

A solid cylindrical waveguide embodiment is illuminated by the same means used for the planar waveguide. The light source, however, is translated along side of the waveguide in a direction parallel to the waveguide's main axis of propagation (the long axis). The entire output of the light source is directed toward the waveguide. This form of illumination is also employed with a hollow cylindrical waveguide (a tubular waveguide). A second embodiment illuminates the tubular waveguide axially through its center (FIG. 8). The illumination waveguide 30 is connected to a light source and movement means at its proximal end. Its distal end is configured so that collimated light traveling through it is emitted equally in a radial direction. Radial emission is accomplished by a 45° bevel on the distal wall of the illumination waveguide, which functions as an annular prism. Upon striking the beveled distal wall, the light makes a 90° turn, taking a trajectory that is directed radially outward (FIG. 9). The outwardly directed light strikes the outer surface of circular waveguide 31, in a ring pattern, and excites any fluorescent rings that might fall in its path.

This form of illumination is a means to create an immersable probe surface. During the processing of a chemical assay, the waveguide is immersed in the analyte solution in a larger enveloping sleeve. This sleeve is either a flexible membrane, bag, or rigid tube. The configuration enables batch processing. This form of illumination also accommodate real time scanning of the probe surface while the chemical assay is taking place. Since illumination waveguide 30 travels inside waveguide 31, it can illuminate waveguide 31 even while immersed in the analyte solution.

Regardless of the embodiment of the waveguide detection system described in this specification, the level of emission from a given spot is linearly dependent upon the intensity of the excitation light to which the spot is exposed. Furthermore, the level of emission from a given spot is effected by the degree of alignment between the excitation light and the probe spot. And, fluorescence from a given probe spot varies inversely with the square of the offset distance between the probe spot and the center of the light.

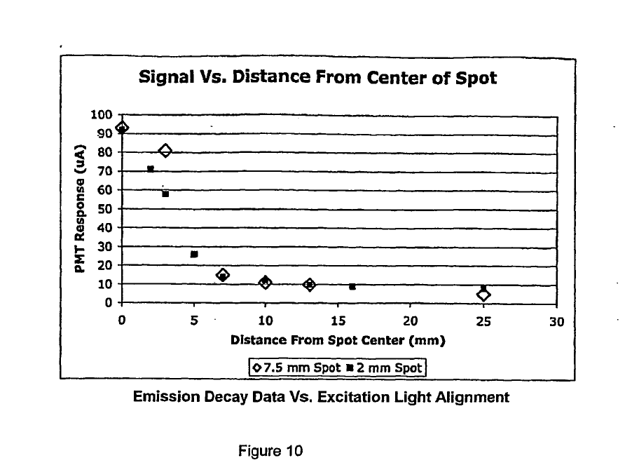

FIG. 10 is a graph of emission decay data as a function of excitation light alignment. It graphs emission data from a waveguide detection system in which the light source is moved relative to the center of a single fluorescent spot immobilized on a 25 mm ×75 mm ×1 mm glass planar waveguide. Emission data was taken from a photo detector aligned at one end of the planar waveguide as shown in FIG. 1. Subsequent emission data was taken as the light source was advanced in the long direction of the waveguide away from the spot. The result shown in the FIG. 10, demonstrates a decay that is a function of the square of the distance between the light source center and the probe spot center. The decaying relationship between emission and alignment with the excitation source provides a means to discriminate between multiple spots on the same waveguide surface. It also provides a means of discriminating between spots that fluoresce simultaneously due to their close proximity.

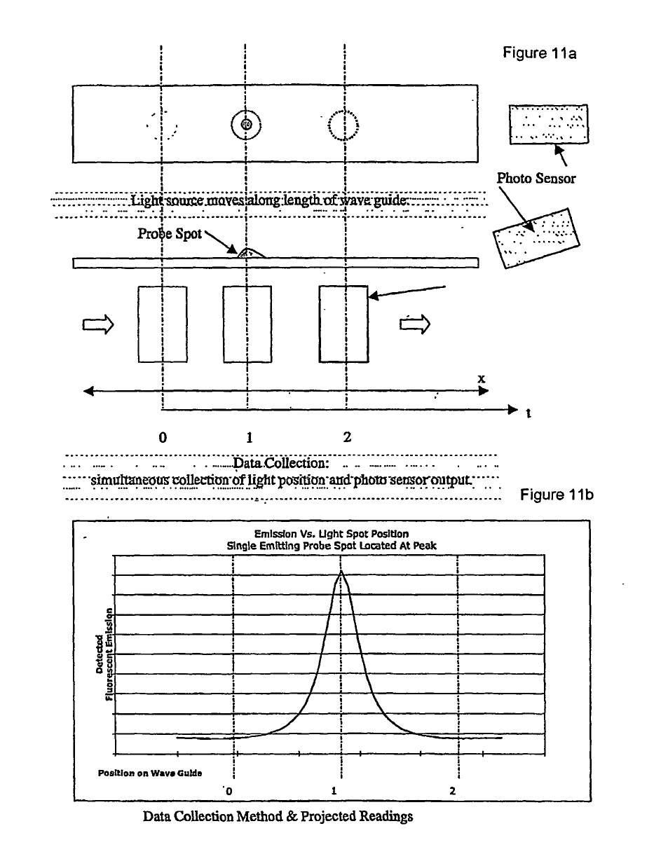

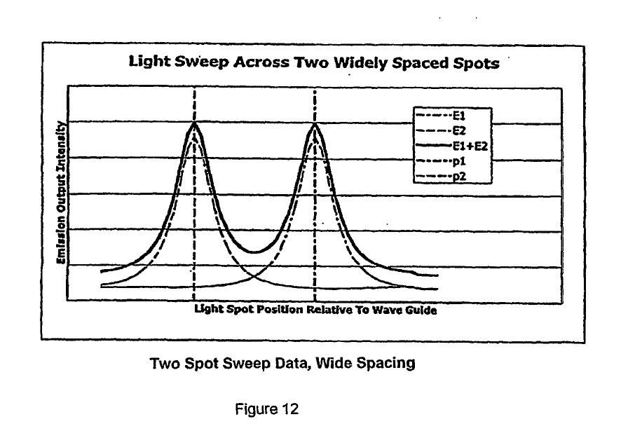

FIG. 11A illustrates the simultaneous determination of light position and the output of the photo detector. FIG. 11B is a graph of the emission intensity from a spot as a function of the position of the light source with respect to the spot. FIGS. 11A and 11B illustrate the fact that if a light source is advanced along a known direction relative to a fluorescently excitable spot, the photo sensor output will vary as a function of the position of the light source relative to the spot on the planar waveguide. FIG. 11A shows the output from one spot on a surface. FIG. 12 is a graph of the output associated with a similar illuminating sweep taken over a surface with two spots. Each of the dotted lines represents the intensity of emission from a flourescent spot that would have occurred if only one or the other of the two spots were fluorescently excitable. The heavy solid line depicts the signal generated if both spots are fluorescent. At the spot spacing chosen for FIG. 12, both spots are distinguishable from one another regardless of their respective fluorescent states. A spacing producing such an output yields distinguishable results by positioning the light source at the loci coincident with the probe spot centers and taking discrete measurements.

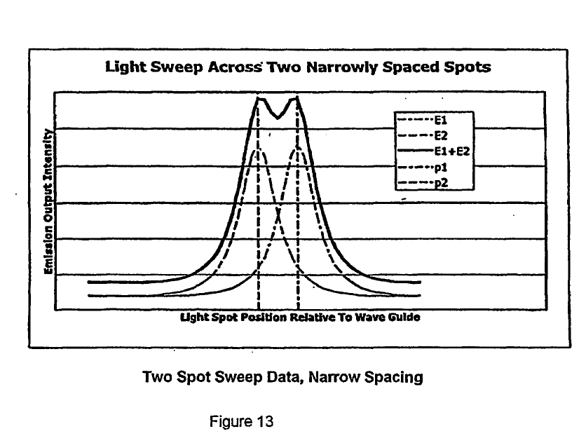

Were the probe spots brought closer together, the combined signals would become more conglomerated and thus more difficult to discretely distinguish from one another ( FIG. 13). With such close spacing, measurements yield ambiguous results. Therefore, a higher number of probe spots (i.e., test sites) yields unreliable results, since it is not clear whether the signal read at a first position comes from fluorescence of the spot at the first position or from a spot at a second adjacent position. However, were a linearly continuous measurement taken, yielding an emission signature as depicted in either FIGS. 12 or 13, any of the possible combinations of fluorescent states among the two probe spots can be distinguished. In FIG. 13, the signature of two adjacent fluorescent spots is distinguishable from the signature of either or neither of the spots fluorescing individually. This same concept can be expanded to inspect arrays of more than 2 spots and to arrays of more than 2 spots and more than one column. In fact, at least 5 or more spots can be detected in a linear space of approximately 25 mm.

The process of detecting fluorescence of closely packed spots comprises the step of moving a single excitation light source in a known trajectory relative to the planar waveguide. Placed upon the planar waveguide are a series of probe spots whose individual fluorescent states are unknown, yet whose loci are well established. Simultaneous and continuous measurements are taken of light source position along the waveguide and the intensity of emission light detected by the photo sensor located at one of the waveguide's ends. The data in FIGS. 11B, 12, and 13 demonstrate that relatively closely packed spots can be distinguished, even when their individual signals interfere with one another. Since the flourescent light intensity for a light spot position signature of combined spots is different from the signature generated from a single spot, the distinction between spots is achievable.

In summary, an embodiment for the multiplexed detection of fluorescence, or lack thereof, and for the intensity of fluorescence on a single planar waveguide surface comprises (i) a series of probe spots bound to specific sites on a planar waveguide, the loci of which are known, (ii) moving a single excitation light source in a known trajectory relative to the planar waveguide, (iii) simultaneously and continuously measuring the light source position along the waveguide, (iv) simultaneously and continuously measuring the intensity of flourescent emission light, or absence thereof, detected by a photo sensor located at one of the waveguide ends, (v) analyzing the continuous intensity waveform output of the photo detector to discriminate between the flourescent light intensity, or lack there, form each individual probe spot in a closely packed waveguide array, (vi) determining which probe spots fluoresce, (vii) relating the fluoresced probe spot to its specific location on the waveguide, and (viii) generating data identifying the genetic material in the sample.

Determination of whether a probe has fluoresced is done by separating the overlapping waveforms from the photo detector output and correlating each separated waveform to a probe binding site. Determining whether a waveform exists for a given spot is a binary identification of whether fluorescence occurred at that spot. The absence of a waveform identifies the correlated probe binding site as non fluorescence. The presence of a waveform identifies the correlated probe binding site as non fluorescence.

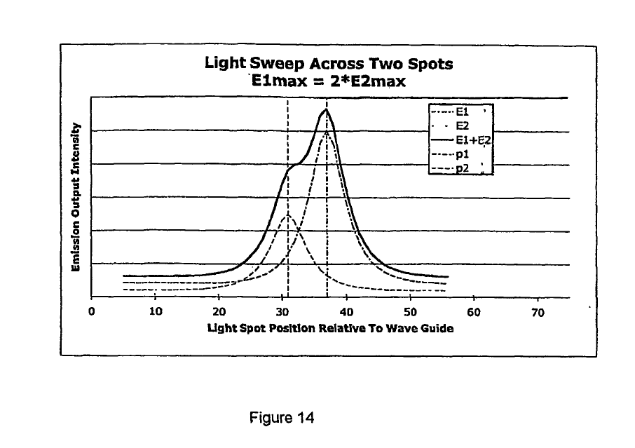

Also of importance is a comparison of the relative intensity of the output of the photo detector for any given probe as compared to that of any other probe spot located on the waveguide surface. Determination of relative intensity allows the clinician to compare the quantity of a target relative to another target or to a control. The photo detector output of the planar waveguide system described in this specification also contains the information necessary to determine the relative flourescent intensity of multiple spots; not just whether the spot is fluorescing. The spots can be distinguished from one another, their relative intensity compared to one another, and the relative concentrations of their associated analytes can be inferred. FIG. 12 illustrates the projected photo sensor response to 2 spots spaced a distance from one another. FIG. 14 illustrates the photo detector response to two spots, one of which produces half the maximum intensity of the other. The solid line in FIG. 14 shows that when a continuous/quasi-continuous measurement is taken, the two spots are distinguishable. Furthermore, their relative intensities can be determined by analyzing the difference in peak height as well as the location and relative magnitude of the inflection point between them.

Although, the multiplexed embodiments described in this specification are capable of detecting multiple analytes in a fluorescent array collocated on the same substrate, if the analyte loci are too close together signal overlap occurs. This high loci density with the concomitant signal waveform overlap may make two analytes indistinguishable from one another. Regardless of the fact that the detector system provides viable genetic testing results in clinical applications, its utility is enhanced by the automated means of determining which spots have been fluorescently activated. In many cases closely spaced spots result in combined waveform signals from the photo detector, yet pose no impediment to visually distinguishing one from the other. However, at the point where the spots are arranged in a denser and/or more complex pattern, their signals combine to such an extent that visual inspection of the graphical output is no longer reliable or convenient.

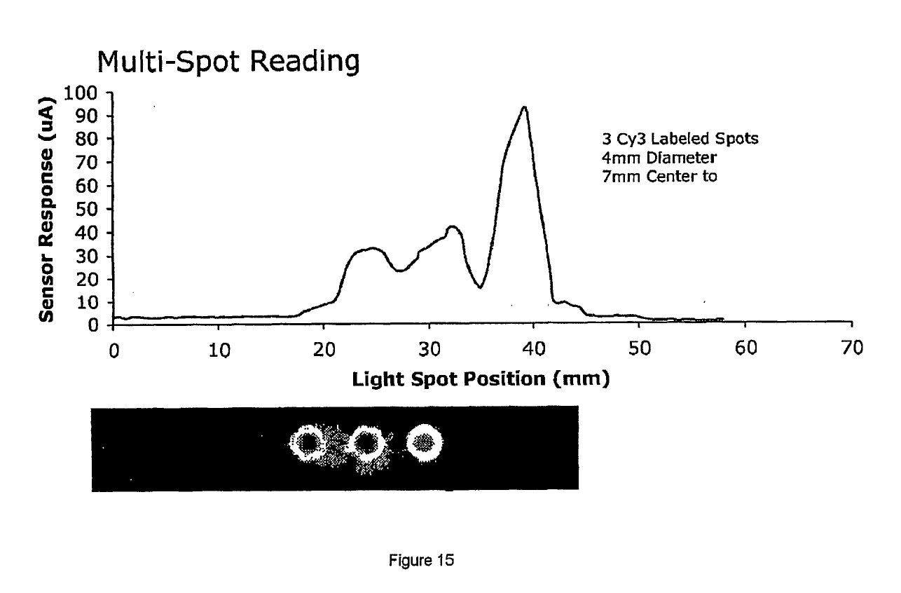

Although, the fluorescence detection output can be resolved by visual inspection of data plotted similarly to that shown in FIG. 15, an automated interpretation of the data is more practical. Automation relieves the user from making determinations by eye, reducing the risk of array misidentification, inconsistency between users, and intensity measurement error. Furthermore, a numerical analysis of data reporting intensity generated from each spot permits a better standard against which the analyst/clinician can make determinations.

An algorithm for determination of the relative intensities of flourescent probes on a waveguide is an element of an embodiment of the detection system described in this specification. There are two versions of the data interpretation algorithm. Both versions resolve signal overlap between spots, determine intensity data of one or more spots, and compare the intensity data of one spot to another spot (test loci). The first algorithm version is an adaptation of the second version. The first algorithm version is used with one dimensional arrays. It is the special case of the second version. The second version is used with two dimensional arrays. A one dimensional array is an array of loci aligned in a single line. A two dimensional array is an array of loci arranged in any two dimensional pattern. The first version of the algorithm uses fewer steps to analyze the data than does the second version because adding another dimension inherently requires additional arithmetic calculation. Nonetheless, the process is the same.

The data interpretation algorithm determines the independent and isolated intensities of all flourescent loci arranged on a surface (e.g., a planar surface, a closed planar surface, a curvilinear surface, a closed curvilinear surface, a planar/curvilinear surface, and a closed planar/curvilinear surface) in an arbitrary, but known pattern. The data interpretation algorithm for the two dimensional array solves a series of simultaneous equations, the quantity of which is equal to the number of array loci, “n.” An embodiment of the data interpretation algorithm is processed by either a special purpose or a general purpose computing device with both the (i) location parameters of the flourescent loci on the surface and (ii) process steps for calculating the relative intensity of any flourescent loci as a function of the output of the photo detector in its memory. The relative intensity for any flourescent loci may be presented on a display unit and/or provided in a printed format. The output of this method is the value of the maximum individual emissions generated by each of the fluorescent spots in the array. Maximum emission for a given fluorescent spot occurs when the excitation light source is aligned with the spot's center. The method requires taking “n+1” measurements inconsistency between users, and intensity measurement error. Furthermore, a numerical analysis of data reporting intensity generated from each spot permits a better standard against which the analyst/clinician can make determinations.

An algorithm for determination of the relative intensities of flourescent probes on a waveguide is an element of an embodiment of the detection system described in this specification. There are two versions of the data interpretation algorithm. Both versions resolve signal overlap between spots, determine intensity data of one or more spots, and compare the intensity data of one spot to another spot (test loci). The first algorithm version is an adaptation of the second version. The first algorithm version is used with one dimensional arrays. It is the special case of the second version. The second version is used with two dimensional arrays. A one dimensional array is an array of loci aligned in a single line. A two dimensional array is an array of loci arranged in any two dimensional pattern. The first version of the algorithm uses fewer steps to analyze the data than does the second version because adding another dimension inherently requires additional arithmetic calculation. Nonetheless, the process is the same.

The data interpretation algorithm determines the independent and isolated intensities of all flourescent loci arranged on a surface (e.g., a planar surface, a closed planar surface, a curvilinear surface, a closed curvilinear surface, a planar/curvilinear surface, and a closed planar/curvilinear surface) in an arbitrary, but known pattern. The data interpretation algorithm for the two dimensional array solves a series of simultaneous equations, the quantity of which is equal to the number of array loci, “n.” An embodiment of the data interpretation algorithm is processed by either a special purpose or a general purpose computing device with both the (i) location parameters of the flourescent loci on the surface and (ii) process steps for calculating the relative intensity of any flourescent loci as a function of the output of the photo detector in its memory. The relative intensity for any flourescent loci may be presented on a display unit and/or provided in a printed format. The output of this method is the value of the maximum individual emissions generated by each of the fluorescent spots in the array. Maximum emission for a given fluorescent spot occurs when the excitation light source is aligned with the spot's center. The method requires taking “n+1” measurements from waveguide 2, one for each of the unknown loci and an-additional measurement of background fluorescence generated by the substrate. If background fluorescence is unimportant to a given analysis, the extra measurement is not necessary and the background value can be assigned an arbitrary value, like 0. Each measurement reports two quantities: (i) current light source position relative to the array and (ii) resulting emission intensity recorded by the photo sensor.

The data interpretation algorithm is described by the expressions set forth, infra, in this specification. The algorithm is based upon three tenets: (i) the location of all potential fluorescent sources is known; (ii) the waveguide output is a linear superposition of the combined fluorescent sources; and (iii) there is a consistent and unique relationship between the emission of a fluorescent source and its position relative to the excitation light. The first tenet is a given. The spots are bonded to the slide in an array that is a precise, pre-established, and measured configuration, which is known. The second tenet is the basis of all micro arrays. Micro array reading depends on a pre-established, consistent, and addressable arrangement of potential test loci. Without it, valid determinations cannot be made. The third tenet is based upon the fact that there are no significant losses associated with transmission within the waveguide. This was demonstrated by taking an asymmetrically placed single spot hybridized to a waveguide surface, holding all other conditions the same, and detecting its emission from both ends of the slide. The result was less than a 1% difference in peak emission between the differently oriented measurements. The numerical data interpretation algorithm is based upon these assumptions. The algorithm estimates the maximum emission for a given excitation from each potential fluorescent source identified.

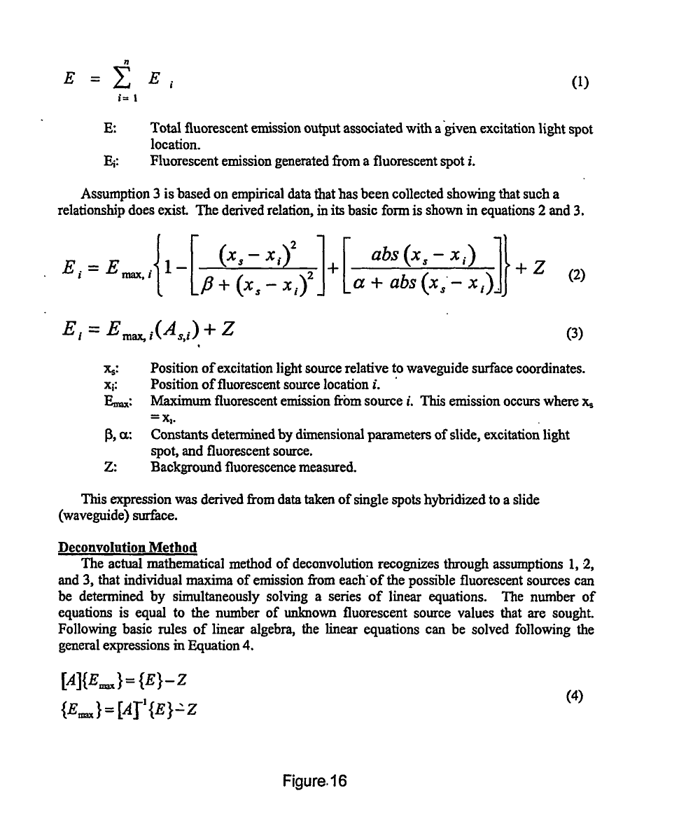

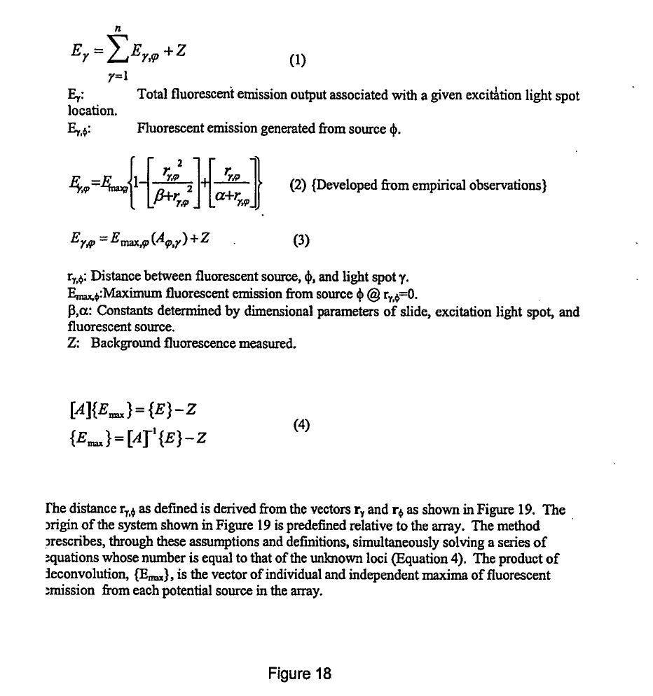

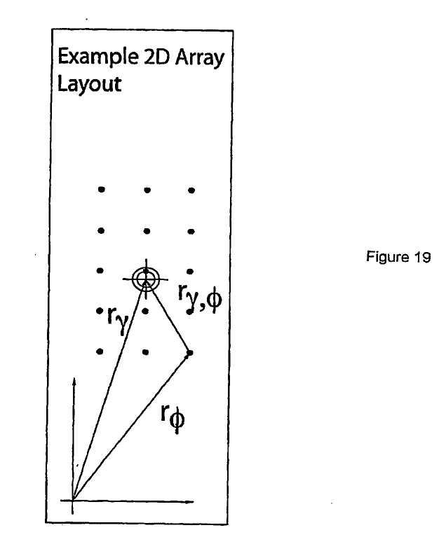

Equations 1 through 4 (FIG. 16) describe the data interpretation algorithm for a linear array of spots on a one dimensional array. Equation 1 expresses tenet 2: the waveguide output is a linear superposition of the combined fluorescent sources the waveguide output is a linear superposition of the combined fluorescent sources. Equations 2 and 3 expresses tenet 3: there is a consistent and unique relationship between the emission of a fluorescent source and its position relative to the excitation light. Equation 4 is based upon assumptions 1, 2, and 3 with recognition that individual maxima of emission from each of the possible fluorescent sources can be determined by simultaneously solving a series of linear equations. The number of equations is equal to the number of unknown fluorescent source values that are sought. Following the basic rules of linear algebra, linear equations 2 and 3 are solved using the general expression of equation 4. It should be noted that a similar deconvolution method has been developed for 2 dimensional arrays. Equation 4 also expresses the generalized solution for a two dimensional array. Equations 1 through 3 (FIG. 18) describe the data interpretation algorithm for a linear array of spots on a two dimensional array. The distance r/gamma and phi, as defined in equation 1 is derived from the vectors r/gamma and r/phi shown in FIG. 19. The origin of the system as shown in FIG. 19 is predefined relative to the array. As with the data interpretation algorithm for a one dimensional array, the algorithm prescribes, based upon the described assumptions and definitions, simultaneously solving a series of equations whose number is equal to that of the unknown loci equation 4 in FIG. 16. The product of {Emax} is the vector of individual and independent maxima of fluorescent emission from each potential source in the array. Determining the vector of individual and independent maxima of fluorescent emission from each potential sources in the array is referred to in this specification as deconvolution of the detector output waveform.

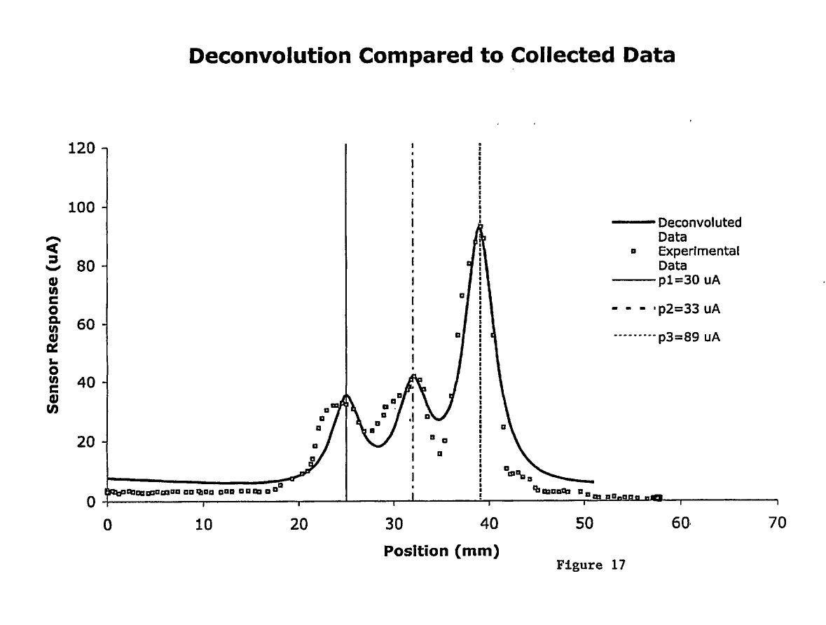

This approach has been validated by following the method discussed above, solving for {Emax} in equation 4 for a one dimensional array, then producing a plot generated from equation 2 for the one dimensional array. This plot is then compared to the data collected from an entire scan of the same array taken by the waveguide system. Typical results of this exercise are shown in FIG. 17. As FIG. 17 suggests, the analytical values are in reasonable agreement with those taken from direct measurement. Correlation between the two data sets is approximately 93%. Coincidence of peak measurements is close, (within 5% of full scale) but there is less concordance in trough areas, within 15% of full scale. The relevance of these differences is highly dependent upon the specific application. Where it is necessary to quantify emission differences between fluorescent regions, this difference may be significant. However, in applications where identifying which areas are emitting and which are not, this disparity is less critical.

Deconvolution ultimately poses advantages over direct visual inspection in a number of aspects. Primarily, it enables a means to report which test loci are emitting a fluorescent signal without direct visual inspection of the data. Furthermore, this method can discriminate between emitting and non-emitting loci in cases where the regions are placed close enough together that their combined signals no longer permit direct visual distinction. In other words this method permits a higher packing density of test sites.

Two dimensional signal deconvolution is used to derive the same vector of peak fluorescent emission values as described for one dimensional signal deconvolution. The method of scanning two dimensional arrays with optical waveguide 2 involves the addition of another degree of freedom of motion and motion tracking for the excitation light source. The two dimensional fluorescent landscape is determined by recording the x position and y position of the illumination source while simultaneously monitoring the output of the photo sensor. Motion can be achieved through manual means, but in an embodiment of the waveguide detector it is achieved by an actuator device. Motion tracking is coupled to the actuator or monitored independently using displacement sensors. As discussed with respect to a one dimensional array, the position of potential fluorescent loci in the array is known because the loci are precisely printed on the array. With the accurate two dimensional tracking of the position of light source 1, as for a one dimensional array, the distance between light source 1 and all array loci is accurately determined for any given recording position of the photo-detector. ‘Based upon the determination of the photo-sensor to array loci distances and associated photo-sensor output, the exact same deconvolution method used for a one dimensional array is employed for a two dimensional array.

One of many genetic tests run on an embodiment of the waveguide detector system is the Factor V Leiden. Factor V Leiden specimens were randomly and blindly chosen from pre-diagnosed clinical samples. They were prepared for solid surface (microscope slide) fluorescence detection. The Factor V Leiden mutation is a single nucleotide change at position 1641 in the gene for the coagulation of factor V. This single nucleotide change results in a condition whereby patients are prone to forming blood clots within veins causing occlusions to blood flow back to the heart. For a given sample two slides were prepared each bearing one probe spot, a wild type and a mutant respectively. Once processed, the slides were scanned by the planar waveguide to determine the presence or absence of either or both the mutant and wild type alleles. Calls made from the planar waveguide were compared to those made by the clinic. All slides were also scanned by the laser scanner and acquisition system, SanArry™ Express Micro array Acquisition System by PerkinElmer™ Life Sciences for secondary verification of the solid surface assay. Factor V Leiden (G1691A) specimens were obtained from the clinical laboratory previously submitted for diagnostic analysis. Specimens were originally diagnosed as having the G to A mutation by Third Wave Technology's (Madison Wis.) Invader (trademark) reaction. In addition samples were confirmed by PCR using FV60 and FV331 factor V Leiden Primers to amplify a 291 bp product.: followed by Ligase detection reaction (LDR). This is considered the standard of comparison for experimental results. Slides were cleaned and then silanized to incorporate a methacrylate moiety. This moiety (or anchor) copolymerized with an overlaid solution of acrylamide, bis-acrylamide, and acrylic acid to create an immobilized gel-pad on the slide surfaces. The acrylic acid functional groups, interspersed throughout the 3D gel-pad matrix, were activated and reacted with amino-modified 24-mer oligonucleotide probes to create a covalent amide bond between probe and surface. Probes were “stenciled” (not spotted as with typical protocols) by adhering a rubber gasket over the surface and delivering probe to the gel-pad. The oligonucleotide probe solution delivered to the surface contained 2% Cy5-fiducial. The scanning of the slide for Cy5 allowed the slides to be quality controlled for confluent probe stenciling and high probe density. The use of gel-pads assures high-density of immobilized probes and high detection signal after subsequent hybridization with ligation detection reactions (LDR) obtained from amplified patient samples.

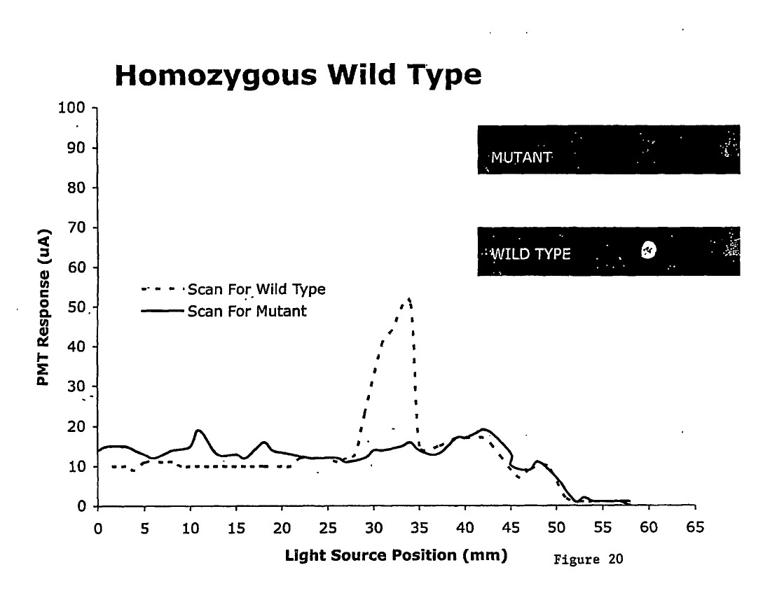

FIG. 20 illustrates a scan for the homozygous wild type and mutant. It plot was done using a planar waveguide detection system embodiment and the process described in this specification. As previously described, a light source moves over the length of a slide (the waveguide) while a photo sensor continuously records florescent emission from one of the slide's ends. The images shown in FIG. 20 along with each of the plots are those of the slides scanned. Images were generated from a micro array slide laser scanner. Two different slides were prepared, one with probes for the mutant sequence, the other with probes for the wild type and the detected data prepared for presentation as FIG. 20. The mutant slide image is dark and no peak is present in the detector data. On the other hand, the homozygous wild type slide has a fluorescent spot and the detector data shows a significant peak at the location of the spot.

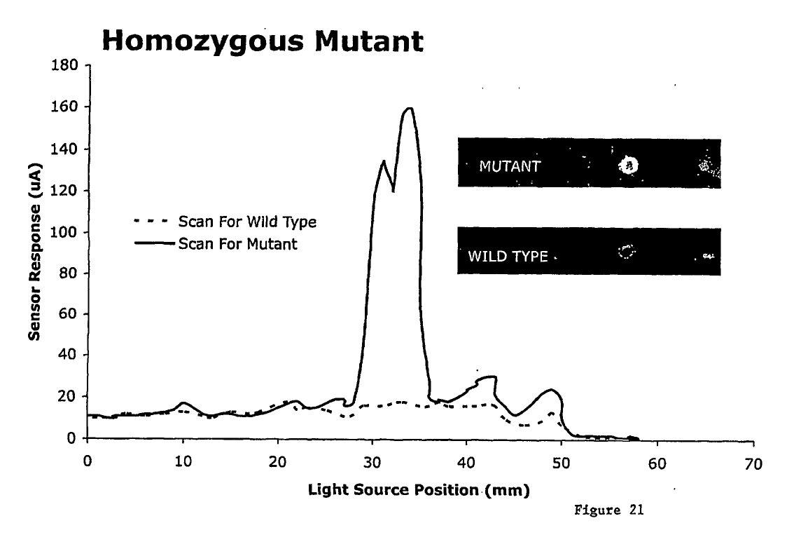

Two different slides were prepared for the homozygous mutant and the detector output is presented in FIG. 21. One slide was prepared with probes for the mutant sequence and the other with probes for the wild type. The wild type slide image is dark and no peak is present in the detector data. On the other hand, the mutant slide has a fluorescent spot and the detector data shows a significant peak at the location of the spot.

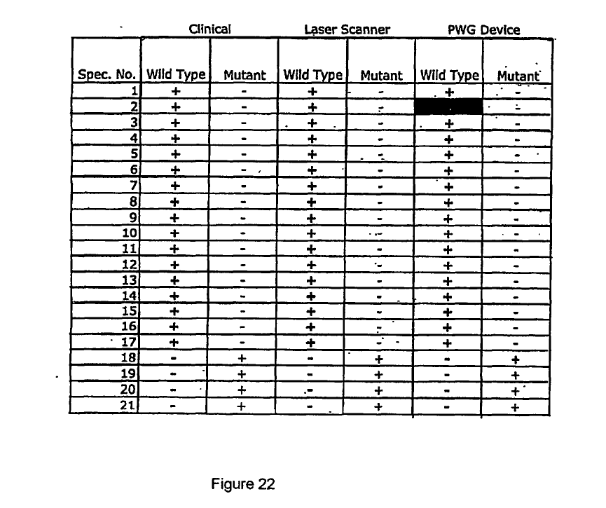

FIG. 22 is a chart of the clinical tests of 21 patients. The chart shows a 98% correlation between calls based on the output of planar waveguide 2 and other detection means.

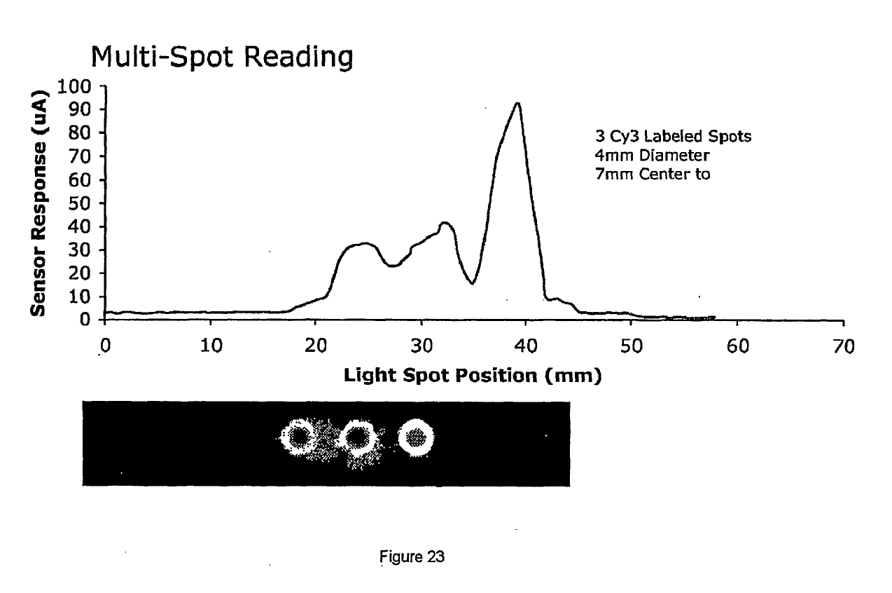

FIG. 23 is a presentation of detector output data from a scan of three spots on the same slide. The third spot from the left in the laser scanner picture of the slide (below the graphed detector output of FIG. 23) is the brightest. The signal detected by a planar embodiment of the waveguide detector reflects that that spot is brightest of the three spots by recording the highest peak. The planar embodiment of the waveguide detector described in this specification provided a quantitative means to compare the intensities of different spots. FIG. 23 illustrates that not only does the waveguide detector distinguish between multiple spots on the same substrate, but it also compares their relative intensities.

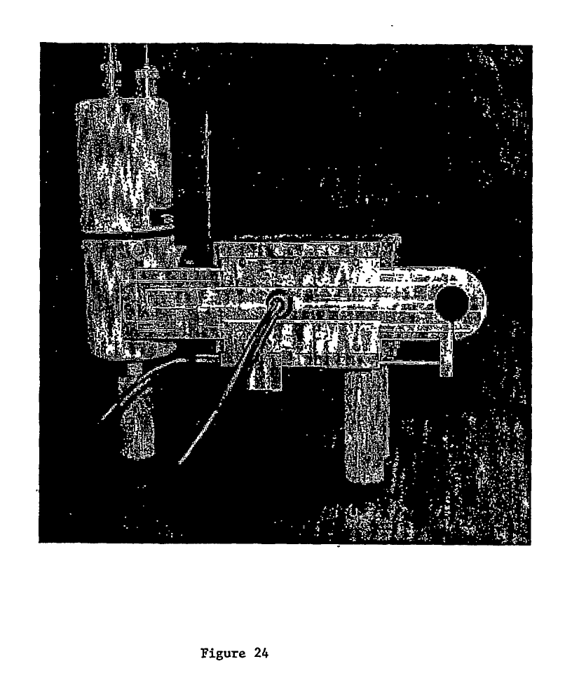

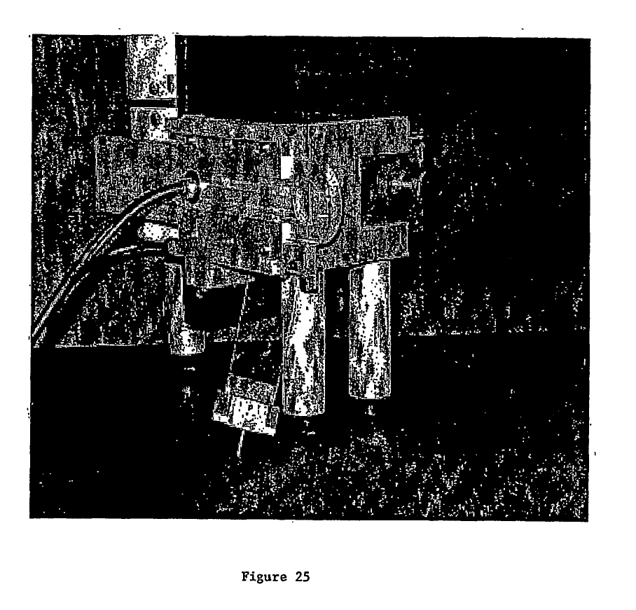

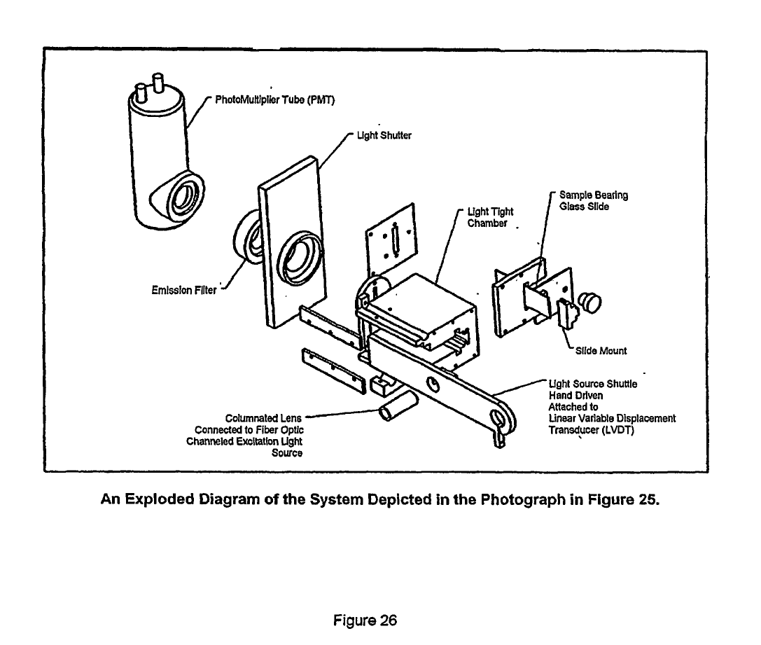

FIG. 24 is a photograph of an embodiment of the waveguide detector from the side. FIG. 25 is a photograph of an embodiment of the waveguide detector from the same side as shown in FIG. 24. However, it is a quartering side view. FIG. 26 is an exploded view of the waveguide detector. Some of the elements of the planar waveguide embodiment and peripheral equipment associated with it is comprised of:
Photo Multiplier Tube (PMT): Hamamatsu R3896 operated at 1000V excitation
PMT Current Amplifier: World Precision Instruments
Light Source: Mill Luce Fiber Optic Illuminator M1000
Filters: Chroma Technology Cy3 Filter Set
Light Shutter: Oriel Instruments
Collimating Lens: Oriel Instruments
LVDT: MLT Displacement Transducer, Honeywell
Data Instruments

An embodiment of the detector also comprises a motion triggered computer data acquisition system using the commercial application LabVIEW™. The system collects simultaneous data output from the amplified photo detector signal and the displacement transducer. It then displays the data in graphical format and saves it for later analysis. The amplified photo detector signal and the displacement transducer outputs are connected to a National Instruments DAQ1200 data acquisition card installed in the PCMCIA slot of a lap top computer.

Various modifications and alterations of the embodiments described in this specification will be apparent to a person of ordinary skill in the art without departing from the scope of the embodiments described nor from the scope of claims set forth in this specification. Furthermore, the claims should not be unduly limited to the illustrative embodiments described in this specification.

We claim:

1. A fluorescent spot detection system comprising (i) an optical waveguide, (ii) excitation light source adapted to move relative to the waveguide over a known interval at an advancement velocity, and (iii) a photo detector statically positioned at an edge of the waveguide, the output of which is continuously or quasi-continuously collected and correlated with the excitation light source position, wherein spot detection is achieved through characterization of the photo detector output relative to the excitation light position.

2. The fluorescent spot detection system of claim 1 also comprising a means for discerning between fluorescent co-located on the same waveguide surface, the close spacing of which spots cause overlap of a fluorescent emission signal from the spots.

3. The fluorescent spot detection system of claim 2, wherein the means for discerning between fluorescent co-located spots is the de-convolution of the composite flourescent emission spectra of the closely spaced spots.

4. The fluorescent spot detection system of claim 3, wherein de-convolution of the composite flourescent emission spectra of the closely spaced spots is taken across the entire field of specific binding site spot loci on the waveguide.

5. The fluorescent spot detection system of claim 4, wherein de-convolution is a function of known fluorescent spot locations.

6. The fluorescent spot detection system of claim 5, also comprising a means for discerning the relative intensity of a fluorescence emission signal between fluorescent spots co-located on a single waveguide surface, the spacing of which may or may not cause overlap of the fluorescent emission signal.

7. The fluorescent spot detection system of claim 6, wherein the means for discerning the relative intensity of a fluorescence emission signal between fluorescent spots co-located on a single waveguide surface is de-convolution of the composite flourescent emission spectra of the closely spaced spots.

8. The fluorescent spot detection system of claim 7, wherein de-convolution of the composite flourescent emission spectra of the closely spaced spots is taken across the entire field of specific binding site spot loci on the waveguide.

9. The fluorescent spot detection system of claim 8, wherein de-convolution is a function of known fluorescent spot locations.

10. A fluorescent diffraction waveguide detector comprising (i) an optical waveguide, (ii) an illumination light source held static relative to the waveguide adapted to provide uniform illumination over the region of the waveguide where fluorescent spots are potentially located, and (iii) a photo detector adapted to move relative to the waveguide over a known interval at a known advancement velocity.

11. The fluorescent diffraction waveguide detector of claim 10, wherein the output from the photo detector is continuously or quasi-continuously collected and correlated with the photo detector's position relative to the waveguide.

12. The fluorescent diffraction waveguide detector of claim 11, wherein spot detection is achieved through characterization of photo detector response relative to photo detector position.

13. The fluorescent diffraction waveguide detector of claim 12, wherein the means for discerning between spots co-located on a single waveguide surface whose spacing is close enough to cause overlap of their respective individual patterns of interference/absorption is the de-convolution of the photo detector response data taken across the entire field of possible spot loci on the waveguide.

14. The fluorescent diffraction waveguide detector of claim 13, wherein de-convolution is based on the prior characterization of known spot arrangements.

15. A fluorescent interference/absorption waveguide detector having a means for discerning relative intensity between spots co-located on the same waveguide surface whose spacing may or may not cause overlap of light interference/absorption.

16. The fluorescent interference/absorption waveguide detector of claim 15, wherein the means for discerning relative intensity between spots is de-convolution of emission response data of possible fluorescent spot loci taken across the entire field of the waveguide.

17. The fluorescent interference/absorption waveguide detector of claim 16, wherein de-convolution of emission response data is based on the prior characterization of known interference/absorption spot arrangements with known relative interference/absorption characteristics.
